(19) United States
(12) Patent Application Publication
(10) Pub. No.: US 2024/0093306 A1
(43) Pub. Date: Mar. 21, 2024

(54) MICRO RNA LIVER CANCER MARKERS AND USES THEREOF

(71) Applicant: THE CLEVELAND CLINIC FOUNDATION, Cleveland, OH (US)

(72) Inventors: Daniel ROTROFF, Pepper Pike, OH (US); Federico AUCEJO, Shaker Heights, OH (US)

(21) Appl. No.: 18/275,797

(22) PCT Filed: Feb. 4, 2022
(86) PCT No.: PCT/US22/15365

§ 371 Date: Aug. 3, 2023

Related U.S. Application Data
(60) Provisional application No. 63/145,718, filed on Feb. 4, 2021.

(51) Int. Cl. C12Q 1/6886; C12Q 1/6874

(52) U.S. Cl. CPC C12Q 1/6886; C12Q 1/6874; C12Q 2600/106; C12Q 2600/178

(57) ABSTRACT

Methods and kits for the detection and treatment of HCC are provided. The methods may be used to distinguish patients having HCC from patients having liver cirrhosis, and comprise detecting differential expression of miRNA relative to a reference sample, such that the HCC patients can be identified and treated accordingly.

### FIELD OF THE DISCLOSURE

The present disclosure relates generally to the use of machine learning to detect differentially expressed microRNA (miRNA) in saliva that are diagnostic biomarkers for hepatocellular carcinoma (HCC).

### BACKGROUND OF THE DISCLOSURE

Liver cancer is the most rapidly increasing cancer in the United States and is estimated to have resulted in 31,780 deaths in 2019 (Cancer Facts & FIGS. 2019. American Cancer Society: Atlanta, GA, USA). Hepatocellular carcinoma (HCC) which is the most common liver cancer, accounts for 80% of all primary liver cancers, and the global incidence of HCC is expected to increase to 78 million by 2030 (Petrick J L, et al. (2016) Journal of Clinical Oncology 34:1787) due to increasing nonalcoholic steatohepatitis (NASH), hepatitis C, and excessive alcohol consumption.

HCC is one of the most aggressive, common neoplasias in the world, characterized by an often unfavorable course. The main therapies with curative potential for cases of hepatocellular carcinoma involve surgical resection or a liver transplant. However, the low postoperative survival rate (30-40% after five years) and frequent post-surgery reappearance of metastasis in patients undergoing a surgical resection treatment considerably complicate the clinical approach toward hepatocellular carcinoma. This limit is further exacerbated by the reduced possibility of surgical treatment, which is in fact restricted to only a small percentage of patients (around 20% of patients with hepatocellular carcinoma), in particular those patients found to have small lesions and relatively normal hepatic parameters.

Early detection of HCC has been shown to improve reception of curative therapy and overall survival (Singal A G, et al. PLoS medicine. 2014;11:e1001624). Unfortunately, however, current HCC serum biomarkers, such as alpha fetoprotein (AFP) and ultrasound, lack prognostic and diagnostic value and result in many false negative diagnoses (Daniele B, et al. (2004) Gastroenterology127:S108-S112; Ayuso C, et al. (2018) European journal of radiology. 101:72-81). Thus, reliable early detection remains elusive.

Furthermore, many assays for HCC do not distinguish between HCC and liver cirrhosis. Liver cirrhosis, which is often a precursor to HCC, has a more hopeful prognosis for patients than HCC because the condition can be arrested or slowed down if the offending condition is stopped or controlled. Therefore, the ability to distinguish between cirrhosis and HCC can be important for patients facing such diagnoses.

As such, there remains a need for improved methods for detection of HCC, and for distinguishing patients having HCC from those having cirrhosis. Fortunately, the following disclosure provides for this and other needs.

### SUMMARY OF THE DISCLOSURE

The disclosure provides salivary miRNA signatures that are highly sensitive and specific non-invasive biomarkers of HCC. Further, the salivary miRNA signatures disclosed herein are differentially abundant in the saliva of patients with HCC compared to the saliva of patients with cirrhosis and are able to distinguish patients having HCC from those having cirrhosis. Therefore, provided are methods, kits, and compositions related to detection of HCC in a patient having or suspected of having HCC.

In accordance with one aspect of the disclosure, there is provided a method for diagnosing or prognosticating hepatocellular carcinoma (HCC) in a subject, or for assessing the risk of developing hepatocellular carcinoma, or for monitoring the effectiveness of an anti-tumor therapy against hepatocellular carcinoma, comprising determining, in an isolated sample of saliva, the expression level of at least one microRNA (miRNA) having at least 90% sequence identity with an miRNA selected from the group comprising or consisting of hsa-mir-148b-3p, hsa-mir-148b-5p, hsa-mir-30d-3p, hsa-mir-30d-5p, hsa-mir-6806-3p, hsa-mir-6806-5p, hsa-mir-6512-3p, hsa-mir-6512-5p, hsa-mir-126-3p, hsa-mir-126-5p, hsa-mir-505-3p, hsa-mir-505-5p, hsa-mir-8059, hsa-mir-193a-3p, and hsa-mir-193a-5p and determining whether the miRNA in the saliva sample is differentially expressed as compared to as compared to a reference saliva sample, wherein the differential expression of miRNA is an upregulation or a downregulation of miRNA expression, in order to determine whether the subject has HCC, is at elevated risk of having HCC, or is receiving effective treatment for HCC, and treating the subject diagnosed with HCC or having elevated risk of having HCC, or as needing further effective treatment for HCC with an a compound or other therapy to improve the HCC.

In one embodiment, the reference sample is a sample from a subject having cirrhosis. In one embodiment, differential expression is detected for at least one miRNA is selected from the group comprising or consisting of hsa-miR-30d-5p, hsa-miR-148b-3p, hsa-mir-6806, and hsa-miR-126-3p as compared to the reference sample. In one embodiment, differential expression is detected for each miRNA in the group comprising hsa-miR-30d-5p, hsa-miR-148b-3p, hsa-mir-6806, and hsa-miR-126-3p as compared to the reference sample, and optionally further comprising one or more of hsa-mir-6512-5p, hsa-mir-505, hsa-mir-8059, and/or hsa-mir-193a-3p. In one embodiment, differential expression is detected for each miRNA of the group comprising or consisting of hsa-mir-148b-3p, hsa-mir-30d-5p, hsa-mir-6806, hsa-mir-6512-5p, hsa-mir-126-3p, hsa-mir-505, hsa-mir-8059, and hsa-mir-193a-3p as compared to a reference expression sample.

In one embodiment, normalized read counts for each miRNA from a small RNA-seq assay are analyzed according to the formula:

Y\hat{ }˜5.371+0.110(hsa-miR-148b-3p)+−5.550(hsa-miR-193a-3p)+−0.441(hsa-mir-8059)+−1.281(hsa-mir-6806)+1.700(hsa-miR-6512-5p)+−0.018(hsa-miR-30d-5p)+−0.037(hsa-miR-126-3p)+0051(hsa-mir-505)
wherein Y\hat{ }=0.44 indicates the subject is at elevated risk of HCC.

In one embodiment, the method has sensitivity of at least 95% and specificity of at least 95% In one embodiment, the method has an accuracy of at least 95% In one embodiment, the reference sample is from a subject who does not have HCC. In one embodiment, the reference sample is from a subject who has cirrhosis.

In accordance with another aspect of the disclosure, there is provided a method for differentiating a subject having hepatocellular carcinoma (HCC) from a subject having liver cirrhosis, the method comprising the step of determining, in an isolated sample of saliva, the expression level of at least one microRNA (miRNA) having at least 90% sequence identity with an miRNA selected from the group comprising or consisting of: hsa-mir-148b-3p, hsa-mir-148b-5p, hsa-mir-30d-3p, hsa-mir-30d-5p, hsa-mir-6806-3p, hsa-mir-6806-5p, hsa-mir-6512-3p, hsa-mir-6512-5p, hsa-mir-126-3p, hsa-mir-126-5p, hsa-mir-505-3p, hsa-mir-505-5p, hsa-mir-8059, hsa-mir-193a-3p, and hsa-mir-193a-5p and determining whether the miRNA in the saliva sample is differentially expressed as compared to a reference saliva sample, wherein differential expression of the miRNA indicates the subject has HCC.

In one embodiment, the at least one miRNA is selected from the group comprising or consisting of hsa-miR-30d-5p, hsa-miR-148b-3p, hsa-mir-6806, and hsa-miR-126-3p. In one embodiment, differential expression is detected for each miRNA in the group comprising hsa-miR-30d-5p, hsa-miR-148b-3p, hsa-mir-6806, and hsa-miR-126-3p as compared to the reference sample, and optionally further comprising one or more of hsa-mir-6512-5p, hsa-mir-505, hsa-mir-8059, and/or hsa-mir-193a-3p. In one embodiment, differential expression is detected for each miRNA of the group consisting of: hsa-mir-148b-3p, hsa-mir-30d-5p, hsa-mir-6806, hsa-mir-6512-5p, hsa-mir-126-3p, hsa-mir-505, hsa-mir-8059, and hsa-mir-193a-3p.

In one embodiment, normalized read counts for each miRNA from a small RNA-seq assay are analyzed according to the formula:

Y\hat{ }˜−5.371+0.110(hsa-miR-148b-3p)+5.550(hsa-miR-193a-3p)+−0.441(hsa-mir-8059)+−1.281(hsa-mir-6806)+1.700(hsa-miR-6512-5p)+−0.018(hsa-miR-30d-5p)+−0.037(hsa-miR-126-3p)-+0.051(hsa-mir-505)
wherein Y\hat{ }=0.44 indicates the subject has HCC.

In one embodiment, the method has sensitivity of at least 95% and specificity of at least 95%. In one embodiment, the method has an accuracy of at least 95%.

In accordance with another aspect of the disclosure, there is provided a method for discovering miRNA biomarkers of hepatocellular carcinoma (HCC) in saliva from a subject having HCC, the method comprising (a) obtaining a saliva sample from the subject, and a saliva sample from a subject not having HCC, and (b) detecting miRNAs that are differentially expressed in the subject having HCC compared to the subject not having HCC, wherein the differentially expressed miRNAs have a high predictive value for detection of HCC, and wherein the detection step comprises machine learning that utilizes least absolute shrinkage and selection operator (LASSO) and cross-validation, wherein differentially expressed miRNAs are miRNA biomarkers of HCC.

In one embodiment, the differentially expressed miRNA biomarkers of HCC are selected from the group comprising or consisting of hsa-mir-148b-3p, hsa-mir-30d-5p, hsa-mir-6806, hsa-mir-6512-5p, hsa-mir-126-3p, hsa-mir-505, hsa-mir-8059, and hsa-mir-193a-3p. In one embodiment, the differentially expressed miRNA biomarkers are selected from the group comprising or consisting of hsa-miR-30d-5p, hsa-miR-148b-3p, hsa-mir-6806, and hsa-miR-126-3p. In one embodiment, the differentially expressed miRNA biomarkers comprise hsa-miR-30d-5p, hsa-miR-148b-3p, hsa-mir-6806, and hsa-miR-126-3p, and optionally further comprise one or more of hsa-mir-6512-5p, hsa-mir-505, hsa-mir-8059, and/or hsa-mir-193a-3p. In one embodiment, the differentially expressed miRNA biomarkers consist of hsa-mir-148b-3p, hsa-mir-30d-5p, hsa-mir-6806, hsa-mir-6512-5p, hsa-mir-126-3p, hsa-mir-505, hsa-mir-8059, and hsa-mir-193a-3p.

In one embodiment, normalized read counts for each miRNA from a small RNA-seq assay are analyzed according to the formula:

Y\hat{ }˜−5.371+0.110(hsa-miR-148b-3p)+5.550(hsa-miR-193a-3p)+−0.441(hsa-mir-8059)+−1.281(hsa-mir-6806)+1.700(hsa-miR-6512-5p)+−0.018(hsa-miR-30d-5p)+−0.037(hsa-miR-126-3p)+0.051(hsa-mir-505)
wherein Y =0.44 indicates the subject has HCC

In one embodiment, the subject not having HCC has liver cirrhosis.

### BRIEF DESCRIPTION OF THE DRAWINGS

FIG. 1. Volcano plots showing the unadjusted log₂ fold change and log P value for A) salivary miRNAs in all patients with HCC (n=20) compared to patients with cirrhosis only (n=19), and B) patients with HCC and chronic liver disease (n=11) compared to patients with cirrhosis only (n=19). Salivary miRNAs with a corresponding log fold change less than −5 and a P<5×10⁻⁶ are annotated using color.

FIG. 2. Heatmap showing the expression of the significant miRNA in each sample (FDR P<0.05 and absolute log₂ fold change >1). Clustering was performed using Ward's D method and Manhattan distance. The dendrogram of samples (columns) was cut to create four clusters, where a distinct cluster with 13 out of 14 samples consisted of HCC samples was observed. The clustering appears to be largely driven by HCC status rather than the presence of chronic liver diseases, such as cirrhosis or fibrosis.

FIG. 3. (A) Venn diagram showing the overlap of miRNAs detected in tissue HCC compared to saliva in patients with HCC versus cirrhosis. In addition, the overlap between miRNA determined to be statistically significantly different between patients with HCC and cirrhosis are shown (FDR P<0.05). (B) The direction of association between saliva and tissue miRNA detected to be statistically significantly different between patients with HCC and cirrhosis across both biospecimens (FDR P<0.05).HCC

FIG. 4. Violin plots (top row) showing the distribution of miRNA in patients with HCC compared to patients with cirrhosis for (A) hsa-miR-148b-3p and (B) hsa-miR-30d-5p. In addition, the receiver operating characteristic curves (ROC) with corresponding area under the curves (middle row) are shown for (C) hsa-miR-148b-3p and (D) hsa-miR-30d-5p. (E) The ROC curves are based on the combination of eight miRNA selected by least absolute shrinkage and selection operator and cross-validation. The three ROC curves represent the predictive accuracy of the model for i) all HCC samples (n=20), ii) HCC samples with chronic liver disease (n=11) and iii) HCC samples without chronic liver disease (n=9).

### DETAILED DESCRIPTION

### Definitions

The term “subject” or ‘patient” as used herein, refers to an individual or mammal having a disease or at elevated risk of having a disease (e.g., having or at elevated risk of having HCC). The “subject” may be diagnosed to be affected by e.g., HCC, or may be diagnosed e.g., to have liver cirrhosis. Similarly, a “subject” may further be diagnosed to be at elevated risk of developing HCC e.g., may have liver cirrhosis. The subject may be any mammal, including both a human and another mammal, e.g. an animal such as a rabbit, mouse, rat, or monkey. Human subjects are preferred. Therefore, the miRNA from a subject may be a human miRNA or a miRNA from another mammal, e.g. an animal miRNA such as a mouse, monkey or rat miRNA, or the miRNAs comprised in a set may be human miRNAs or miRNAs from another mammal, e.g. animal miRNAs such as mouse, monkey or rat miRNAs.

The expression “patient not having HCC” as used herein, may refer to either to a patient per se or to a reference data set such as microarray expression data from a normal patients or microarray expression data from patient(s) having liver cirrhosis.

The term “reference level,” “reference sample,” “control level,” “control sample,” or grammatically equivalent expressions are used interchangeably herein to refer to a reference sample to which a test sample from a subject is compared. The nature of the reference sample depends on the particular diagnosis to be made. For example, to determine if a subject having liver cirrhosis also has HCC or is at elevated risk of developing HCC, the “reference level,” or “reference sample” may be miRNA sequence data or microarray data from a subject known to have cirrhosis, but not HCC. Alternatively, a “reference sample” may be miRNA sequence data or microarray data from a healthy subject without HCC or any cancer related diseases. Appropriate controls are readily chosen by a person having ordinary skill in the art.

The term “differentially expressed” or “differential expression” as used herein refers to microRNAs (miRNA) which differ in abundance between a test sample and a reference sample or control, for example which differ in abundance between a patient having HCC and a patient having cirrhosis. miRNAs are differentially expressed when their expression levels are either higher or lower than expression in a reference sample or control. The degree to which miRNA expression differs need only be large enough to be quantified via standard expression characterization techniques. Thus, “differential expression” can be determined by any method known in the art, such as by array hybridization, next generation sequencing, RT-PCR and other methods as would be understood by a person skilled in the art, e.g. by quantitative hybridization (e.g. to a microarray, to beads), amplification (PCR, RT-PCR, qRT-PCR, high-throughput RT-PCR), next generation sequencing (e.g. ABI SOLID, Illumina Genome Analyzer, Roche 454 GS FL) and the like.

As used herein, the term “miRNA” or “microRNA” or “miR” refers to an RNA (or RNA analog) comprising the product of an endogenous, non-coding gene whose precursor RNA transcripts can form small stem-loops from which mature “miRNAs” are cleaved by the endonuclease Dicer. MiRNAs are encoded in genes distinct from the mRNAs whose expression they control. In one example, the term “miRNA” or “microRNA” refers to single-stranded RNA molecules of at least 10 nucleotides and of not more than 35 nucleotides covalently linked together. In one example, the polynucleotides are molecules of 10 to 33 nucleotides or of 15 to 30 nucleotides in length, or of 17 to 27 nucleotides or of 18 to 26 nucleotides in length, i.e. 10, 11, 12, 13, 14, 15, 16, 17, 18, 19, 20, 21, 22, 23, 24, 25, 26, 27, 28, 29, 30, 31, 32, 33, 34, or 35 nucleotides in length, not including optionally labels and/or elongated sequences (e.g. biotin stretches). The sequences of the miRNAs as disclosed herein include, but are not limited to miRNA sequences SEQ ID NO: 1 to SEQ ID NO: 16.

As will be appreciated in the art of the present disclosure, in any of the methods as disclosed herein, the miRNAs may not have 100/a sequence identity with the sequences of miRNAs as disclosed herein. Thus, in one example, the measured miRNA may have at least 75%, or at least 80%, or at least 85%, or at least 90%, or at least 95%, or at least 97.5%, or at least 98%, or at least 99%, or at least 99.9% sequence identity to the miRNAs disclosed herein. In one example, the measured miRNAs may have one, two, three or four nucleotide substitutions. Thus, the miRNAs as used in the methods disclosed herein may be detected using reagents that may be able to hybridize or bind specifically to the disclosed sequences (e.g., SEQ ID NOs 1-16). As used herein, the terms “hybridizing to” and “hybridization” are interchangeable used with the terms “specific for” and “specifically binding” and refer to the sequence specific non-covalent binding interactions with a complementary nucleic acid, for example, interactions between a target nucleic acid sequence and a target specific nucleic acid primer or probe. In one example, a nucleic acid probe, which hybridizes is one which hybridizes with a selectivity of greater than 70%, greater than 80%, greater than 90% or of 100% (i.e. cross hybridization with one of the miRNAs as disclosed herein may occur at less than 30%, less than 20%, less than 10%). As would be understood to a person skilled in the art, a nucleic acid probe, which “hybridizes” to the miRNA as disclosed herein may be determined taking into account the length and composition. In one example, the nucleic acid probes, which hybridize with the any of the miRNAs as disclosed herein may have one, or two, or three mismatched base pairing. Thus, the miRNA disclosed herein may include miRNAs which may differ from SEQ OID NOs 1-16 by 1, 2, 3, 4, or 5 nucleotides.

The term “accuracy” as used herein, has the meaning commonly understood in the art (see e.g., Fawcett, Tom (2006) Pattern Recognition Letters. 27 (8): 861-874) and refers to the degree of closeness of measurements of a quantity to that quantity's true value, and is calculated as the sum of true positives plus true negatives divided by the sum of all positives and all negatives.

The term “sensitivity” as used herein has the meaning commonly understood in the art (see e.g., Fawcett, (2006) supra). “Sensitivity” is a statistical measure of how well a binary classification test correctly identifies a condition, and refers to the ability of the analytical method or algorithm to truly determine the individuals that have the disease. Thus, sensitivity is a measure of how well a test can identify true positives. As known in the art (Yerushalmy, J. (1947) Public Health Reports. 62 (2): 1432-39; Fawcett, Tom (2006) Pattern Recognition Letters. 27 (8): 861-874; Powers, David M W (2011) Journal of Machine Learning Technologies. 2 (1): 37-63), sensitivity measures the proportion of positives that are correctly identified (e.g. the proportion of those who have HCC who are correctly identified as having the condition). Thus, Sensitivity=True Positive/(True Positive+False Negative)×100%.

The term “specificity” as used herein has the meaning commonly understood in the art (see e.g., Fawcett, (2006) supra). “Specificity” is a statistical measure of how well a binary classification test correctly identifies a condition, for example how frequently it correctly classifies a subject having HCC or at elevated risk of developing HCC. “Specificity” measures the proportion of negatives that are correctly identified (e.g. the proportion of those who do not have HCC who are correctly identified as not having HCC). Thus, Specificity=True Negative/(False Positive+True Negative)×100% or 1-false positive rate.

A discussion of “sensitivity” and “specificity” as known in the art can be found, for example, on the world wide web at en.wikipedia.org/wiki/Sensitivity_and_specificity.

The term “predictive value” or “positive predictive value” as used herein refers to the ratio of true positives out of all identified positives.

The term “Receiver operating characteristic (ROC) curves” refers to a graphical measure of sensitivity (y-axis) vs. 1-specificity (x-axis) for a clinical test, which is known in the art (see e.g., Fawcett, (2006) supra). A measure of the accuracy of a clinical test is the area under the ROC curve value (AUC value). If this area is equal to 1.0 then this test is 100% accurate because both the sensitivity and specificity are 1.0 so there are no false positives and no false negatives. On the other hand a test that cannot discriminate is the diagonal line from 0,0 to 1,1. The ROC area for this line is 0.5. ROC curve areas (AUC-values) are typically between 0.5 and 1.0. Thus, an AUC-value close to 1 (e.g. 0.95) represents a clinical test as that has high sensitivity and specificity and accuracy.

The term “biomarker” as used herein, refers to a characteristic that can be objectively measured and evaluated as an indicator of normal and disease processes or pharmacological responses. A “biomarker” is a parameter that can be used to measure the onset or the progress of disease or the effects of treatment. The parameter can be chemical, physical or biological.

### I. Introduction

Current approaches used to screen patients for HCC lack sensitivity and accuracy, resulting in too any false negative diagnoses. Furthermore, in many cases it is not possible to distinguish patients who are experiencing HCC and cirrhosis from those who experience cirrhosis only. Accordingly, there is a significant need for more sensitive screening tools to detect HCC that would allow for early diagnosis, and additionally that can distinguish whether or not a patient is experiencing cirrhosis alone, or cirrhosis and HCC. Ideally, such screening tools would also be non-invasive and cost-effective so that they are easy to use and widely accessible, thus improving patient outcome.

MicroRNAs (miRNAs) are small, non-coding RNA molecules that regulate gene expression by translational inhibition or mRNA degradation. MiRNAs are naturally present in many organisms, including animals, plants and viruses, and play a fundamental role in the control of gene expression by regulating, in a specific manner, the stability and translation of messenger RNAs (mRNAs). MiRNAs are initially expressed as long precursor RNA molecules, or pri-miRNAs, which by means of a complex mechanism of nucleo-cytoplasmic processing, are transformed into the mature form (miRNA), characterized by a length of 17-24 nucleotides. The function of many miRNAs is not known. However, various studies have demonstrated the key role that miRNAs have in gene regulation in many fundamental biological functions such as apoptosis, hematopoietic development and cellular differentiation.

Although the majority of miRNA regulation occurs intracellularly, in some cases select miRNAs are found in the circulation and have been associated with numerous human diseases such as cancer, NASH/NAFLD and alcoholic hepatitis (Eslam M, et al. (2018) J. Hepatology. 68:268-279; Blaya D, et al. (2016) Gut. 65:1535-1545) and patient survival (Dongiovanni P, et al.(2018) International journal of molecular sciences 19:3966; Pineau P, et al. (2010) Proceedings of the National Academy of Sciences 107:264-269).

Differentially expressed miRNA in blood have been detected in HCC (Murakami Y, et al. PloS one. 2012;7; Qi J, et al. (2013) Neoplasma 60:135). However, before this disclosure, it was not known that particular detectable alterations in the expression of salivary miRNAs were present in patients with HCC and that these alterations are indicative of the fact that the subject is affected by hepatocellular carcinoma. Furthermore, prior to this disclosure, it was not known that alterations in the expression of particular salivary miRNAs could be used to distinguish patients experiencing liver cirrhosis from those experiencing HCC.

### II. General Methods

A patient suspected of having HCC can be identified by any method known in the art. A patient suspected of having HCC can be identified by behavioral or experiential circumstances or by physical or clinical symptoms. For example, the risk of HCC is typically higher in people with long-term liver diseases. Thus, patients experiencing hepatitis B or hepatitis C may have or be suspected of having HCC. HCC is also more common in people who drink large amounts of alcohol, who take certain drugs such as e.g., anabolic steroids, who have too much iron stored in the liver, who experience exposure to aflatoxins, and/or individuals who have an accumulation of fat in the liver such as individuals who are obese or who have diabetes.

Typically, early stages of HCC do not present any symptoms. Thus, determining whether a patient is suspected of having HCC may be made by a physician based on patient history. However, later stages of HCC often exhibit symptoms such as e.g., upper abdominal pain, weight loss, jaundice, fluid in the abdomen, and/or liver failure.

This disclosure utilizes routine techniques in the field of recombinant genetics. Basic texts disclosing the general methods and terms in molecular biology and genetics include e.g., Sambrook et al., Molecular Cloning, a Laboratory Manual, Cold Spring Harbor Press 4th edition (Cold Spring Harbor, N.Y. 2012); Current Protocols in Molecular Biology Volumes 1-3, John Wiley & Sons, Inc. (1994-1998). This disclosure also utilizes routine techniques in the field of biochemistry. Basic texts disclosing the general methods and terms in biochemistry include e.g., Lehninger Principles of Biochemistry sixth edition, David L. Nelson and Michael M. Cox eds. W.H. Freeman (2012).

This disclosure also utilizes routine methods in the fields of statistics and machine learning. Basic texts disclosing the general methods and terms statistics and machine learning include e.g., Fawcett, Tom (2006) Pattern Recognition Letters. 27 (8): 861-874; Encyclopedia of Machine Learning and Data Mining, Claude Sammut, and Geoffrey I. Webb, eds. Springer (2017) and The Elements of Statistical Learning: Data Mining, Inference, and Prediction, Trevor Hastie, Robert Tibshirani, and Jerome Friedman, eds. 2^{nd} Edition Springer (2017).

This disclosure also utilizes routine methods in the field of bioinformatics. Basic texts disclosing the general methods and terms in bioinformatics include e.g., Current Protocols in Bioinformatics, Andreas D. Baxevanis and Daniel B. Davison eds. Wiley (2003). Known miRNA sequences can be found in the miRBase v22 (Griffiths-Jones S. (2010) Current protocols in bioinformatics 29:12-9), which is located on the world wide web at mirbase.org. “miRBase” refers to a well-established repository of validated miRNAs. The miRBase is a searchable database of published miRNA sequences and annotation. Each entry in the miRBase Sequence database represents a predicted hairpin portion of a miRNA transcript (termed mir in the database), with information on the location and sequence of the mature miRNA sequence (termed miR). Both hairpin and mature sequences are available for searching and browsing, and entries may be retrieved by name, keyword, references and annotation. All sequence and annotation data are also available for download.

### Sample Collection and Data Generation

Saliva sample can be collected from patients using any method known in the art. miRNA isolated from saliva is quantitated and analyzed by any methods known in the art, for example using small RNA sequencing (RNA-Seq see e.g., Wang Z, Gerstein M, Snyder M. (2009) Nat Rev Genet. 2009; 10(1):57-63) qPCR (see e.g., Biochem (Lond) (2020) 42 (3): 48-53) etc.

Differential Expression of miRNA

Though well-codified, generally accepted methods for classifying the pathological progression of many forms of tumors are available today. However, the clinical classification of hepatocellular carcinoma and the correlated therapeutic indications entail very complex procedures and depend both on the degree of tumor progression and residual liver function and thus, it can be difficult to accurately determine if an individual has HCC and/or to distinguish whether a high risk patient such as an individual having cirrhosis, has HCC. Therefore, of particular interest, and currently unmet need, is the ability to detect HCC in high-risk individuals, such as those with cirrhosis. Therefore, the identification of specific markers capable of accurately screening patients affected by hepatocellular carcinoma and distinguishing those patients from individuals having liver cirrhosis is an indispensable objective, especially because the ability to intervene when the disease is early can maximize the chance of patients being eligible for curative treatments.

The objective of a universally accepted staging is potentially useful for improving the accuracy of the prognosis in individual patients, favoring the selection of patients for different therapies and, finally, adapting groups of patients based on therapeutic efficacy.

The identification of molecular biomarkers offers hope of improving the diagnosis or prognosis of hepatocellular carcinoma, assessing the risk of developing hepatocellular carcinoma and monitoring the effectiveness of a therapeutic treatment against hepatocellular carcinoma.

In this context, the technical problem at the basis of the present disclosure is to provide a method for detecting hepatocellular carcinoma and distinguishing HCC from liver cirrhosis. The method is not invasive, is simple and fast, and at the same time accurate and reproducible, and is useful for assuring the choice of the best therapeutic treatment for each individual patient. For example, the method can be a factor in determining if a patient should be treated for HCC or not or can be taken into consideration when deciding what follow-up tests should be done (e.g., biopsy), defining the response to therapies, monitoring any possible recurrences of the hepatocellular carcinoma, and identifying new therapeutic targets.

In the context of the present disclosure, the term “non-invasive” signifies the possibility, by means of a simple saliva test, of devising made-to-measure treatments for individual patients, as opposed to relying on disadvantageous methods with costly imaging and invasive biopsies, which at present represent the classic clinical approach for cancer diagnosis, prognosis and hence therapy. In particular, a specific panel of biomarkers, present and stable in the saliva, can be used as a molecular “fingerprint” of hepatocellular carcinoma.

The present disclosure relates to a method for diagnosing or prognosticating hepatocellular carcinoma, also in the early stages, for assessing the risk of developing hepatocellular carcinoma or for monitoring the effectiveness of an anti-tumor therapy against hepatocellular carcinoma, which comprises measuring, for example by quantitative RT-PCR or Small RNA sequencing (RNA-Seq, see e.g., Wand, et al. (2009) Nat Rev Genet. 10(1): 57-63), the expression level of at least one microRNA (miRNA) in a saliva sample and comparing said measured expression level with a reference level.

In an exemplary embodiment, the disclosure provides eight (8) miRNAs (or the complements thereof) that are differentially expressed in a subject having HCC compared to a reference level (e.g., a subject not having HCC).

SEQ ID NO: 1 (hsa-mir-148b-3p): UCAGUGCAUCACAGAACUUUGU or the complement thereof, SEQ ID NO: 2 (hsa-mir-148b-5p): AAGUUCUGUUAUACACUCAGGC

SEQ ID NO: 3 (hsa-mir-30d-3p): CUUUCAGUCAGAUGUUUGCUGC or the complement thereof, SEQ ID NO: 4 (hsa-mir-30d-5p): UGUAAACAUCCCCGACUGGAAG;

SEQ ID NO: 5 (hsa-mir-6806-3p): UGAAGCUCUGACAUUCCUGCAG or the complement thereof SEQ ID NO: 6 (hsa-mir-6806-5p):

UGUAGGCAUGAGGCAGGGCCCAGG

SEQ IN NO: 7 (hsa-mir-6806)

UGCUCUGUAGGCAUGAGGCAGGGCCCAGGUUCCAUGUGAUGCUGAAGCUC
UGACAUUCCUGCAG

SEQ ID NO: 8 (hsa-mir-6512-3p): UUCCAGCCCUUCUAAUGGUAGG or the complement thereof SEQ ID NO: 9 (hsa-mir-6512-5p):UACCAUUAGAAGAGCUGGAAGA

SEQ ID NO: 10 (hsa-mir-126-3p)UCGUACCGUGAGUAAUAAUGCG or the complement thereof SEQ ID NO: 11 (hsa-mir-126-5p): CAUUAUUACUUUUGGUACGCG

SEQ ID NO: 12 (hsa-mir-505-3p): CGUCAACACUUGCUGGUUUCCU or the complement thereof SEQ ID NO: 13 (hsa-mir-505-5p): GGGAGCCAGGAAGUAUUGAUGU

SEQ ID NO: 14 (hsa-mir-8059):

UACAGGUGCAGGGGAACUGUAGAUGAAAAGGCUUGGCACUUGAGGGAAAG
CCUCAGUUCAUUCUCAUUUUGCUCACCUGUU

SEQ ID NO: 15 (hsa-mir-193a-3p) AACUGGCCUACAAAGUCCCAGU or the complement thereof SEQ ID NO: 16 (hsa-mir-193a-5p): UGGGUCUUUGCGGGCGAGAUGA

Thus, differential expression of one or more of miRNAs according to SEQ ID NOs 1-16 is indicative of HCC. In some embodiments, differential expression of each of SEQ ID NOs 1-16 compared to a reference level distinguishes a subject having HCC from a subject having liver cirrhosis.

Differential expression of miRNA compared to a reference level can be determined by any method known in the art (see e.g., Foye, Catherine, et al. PLoS One 12.12 (2017): e0189165; Tian, T. et al., (2015) Org. Biomol. Chem. Feb 28;13(8):2226-38) including, but not limited to use of next generation sequencing, DNA-gold nanoparticle probes (Degliangeli F. et al.,(2014) J. Am. Chem Soc. Feb 12;136(6):2264-7), and quantitative PCR (Cirera S, and Busk P K.(2014) Methods Mol Biol. 1182:73-81)

An alteration in the expression levels of a miRNA in a sample of a test subject, as compared to a control sample, is indicative of the fact that the subject is affected by hepatocellular carcinoma or has an increased risk of developing hepatocellular carcinoma. Furthermore, an alteration in the expression levels of an miRNA in a sample of the test subject, as compared to a control sample, is indicative of the effectiveness, evolution and outcome of a therapy against hepatocellular carcinoma.

An alteration in the expression levels of a miRNA in a sample of the test subject, as compared to a control sample, may also be indicative of the evolution of the disease and hence of the prognosis thereof.

The methods disclosed herein can also be used to diagnose or assess the risk of developing HCC in liver cirrhosis patients affected, for example, by chronic hepatitis or in healthy subjects, or to prognosticate the evolution of cirrhosis in patients affected by cirrhosis, or to monitor the effectiveness of a pharmacological therapy against liver cirrhosis or to monitor the effectiveness of a pharmacological therapy to prevent or mitigate HCC.

In an exemplary embodiment, the method comprises measuring, for example by quantitative RT-PCR or RNA-Seq, the expression level of at least one microRNA (miRNA) in a saliva sample and comparing said measured expression level with a reference level. An alteration in the expression levels of a miRNA gene product in a sample of the test subject, as compared to a control sample, is indicative of the fact that the subject is affected by HCC or has an increased risk of developing HCC, as for example in the case of patients affected by liver cirrhosis.

Such alteration is also indicative of the effectiveness, evolution and outcome of a therapy against liver cirrhosis to prevent further development to HCC.

The methods disclosed herein can be applied in combination with: microarrays, proteomic and immunological analyses, and sequencing analyses of specific DNA sequences for the purpose of defining an ad hoc therapeutic or diagnostic approach for individual patients. Completing the clinical information derived from known investigative techniques with that of the present disclosure would help to address the treatment of a patient affected by hepatocellular carcinoma or cirrhosis in a completely personalized manner that is advantageous as regards both the diagnosis and the prognosis and therapy.

Differentially expressed miRNAs as disclosed herein, are useful as biomarkers for identifying the pathology, defining the response to therapies and monitoring any possible recurrences of the hepatocellular carcinoma. Such miRNAs are also useful for defining the altered molecular pathways in hepatocellular carcinoma and can contribute, therefore, to identifying new therapeutic targets.

### Machine Learning and Artifcial Intelligence

Machine learning methods can be used to model the likelihood of that miRNA is predictive of the presence of HCC. Least absolute shrinkage and selection operator (LASSO) penalized logistic regression is an 11-penalized regression method that performs both regularization and variable selection, which results in a regression solution with improved interpretability and prediction accuracy compared to other regression approaches. LASSO is known in the art (see e.g., Tibshirani R. et al. J R Stat Soc Series B Stat Methodol. 1996; 1:267-88).

Cross-validation may be used to evaluate predictive models by partitioning the original sample into a training set to train the model, and a test set to evaluate it. Cross-validation, is any of various model validation techniques for assessing how the results of a statistical analysis will generalize to an independent data set. Typically, a model is given a dataset of known data on which training is run (training dataset), and a dataset of unknown data against which the model is tested (called the validation dataset or testing set). The goal of cross-validation is to test the model's ability to predict new data that was not used in estimating it to give an insight on how the model will generalize to an independent dataset. Cross-validation is known in the art (see e.g., The Elements of Statistical Learning: Data Mining, Inference, and Prediction. By Trevor Hastie, Robert Tibshirani, and Jerome Friedman, Second Edition, Springer 2009.

Thus, in order to discover miRNA biomarkers that can discriminate, for example, between two or more clinical conditions, e.g. HCC and cirrhosis, the inventors applied a machine learning approach (e.g. LASSO,Ten-fold cross-validation, area under the receiver operating characteristic curve (AUC), sensitivity, specificity, positive predictive value (PPV), negative predictive value (NPV), and balanced accuracy) which leads to an algorithm that is trained by reference data (i.e. data of reference miRNA expression profiles from the two clinical conditions, e.g. HCC and cirrhosis, for the defined set of miRNA markers) to discriminate between the two statistical classes (i.e. two clinical conditions, e.g. HCC and cirrhosis).

As will be discussed in detail below, the inventors have thus discovered particular miRNAs that alone or in combination provide high diagnostic accuracy, specificity and sensitivity in the determination of HCC in patients. Said miRNA are preferably selected from the group consisting of SEQ ID NO: 1 to SEQ ID NO: 16.

### Kits

In some embodiments, the disclosure provides kits comprising instructions for analyzing differentially expressed miRNAs as disclosed herein.

### Example

The following Example illustrates methods for detecting miRNA that are differentially expressed in saliva samples from subjects having HCC, and using the differentially expressed miRNA to distinguish patients having HCC and patients having cirrhosis.

### Methods

### Patient Recruitment and Sample Collection

Saliva samples were collected from 20 individuals with HCC and 19 from individuals with cirrhosis seen at the Cleveland Clinic (Cleveland, OH). Participants were adult patients (>18 years of age) who underwent liver transplantation for HCC, surgical resection for liver tumors or liver biopsy. A description of the cohort is given in Table 1, including the frequencies of chronic liver disease between the two groups. Eleven out of the twenty patients with HCC had chronic liver disease, and analyses were performed with these samples combined and stratified by liver disease, as described below. Initial disease diagnoses were made from a combination of clinical presentation, imaging and laboratory techniques. Subsequently, these diagnoses underwent a secondary confirmatory pathological diagnosis. All participants provided written informed consent and the study was approved by the Cleveland Clinic IRB.

TABLE 1
Summary statistics for the study cohort.

| | Cirrhosis | | | |
|---|---|---|---|---|
| Characteristic | without HCC | | HCC | |
| Total (N) | 19 | | 20 | |
| Mean Age (min-max) | 57.2 | (33-80) | 67.9 | (53-89) |
| Sex | | | | |
| Male (%) | 9 | (47%) | 14 | (70%) |
| Female (%) | 10 | (53%) | 6 | (30%) |
| Race | | | | |
| Caucasian (%) | 18 | (95%) | 10 | (50%) |
| Black (%) | 0 | (0%) | 0 | (0%) |
| Hispanic (%) | 1 | (5%) | 1 | (5%) |
| Unspecified (%) | 18 | (95%) | 9 | (45%) |
| Mean BMI (min-max) | 33.12 | (21.07-57.96) | 29.44 | (19.53-41.8) |
| Chronic Liver Disease | 19 | (100%) | 11 | (55%) |
| Fibrosis (%) | 0 | (0%) | 2 | (10%) |
| Cirrhosis (%) | 19 | (100%) | 9 | (45%) |
| NASH (%) | 7 | (37%) | 5 | (25%) |
| EtOH^{a} (%) | 7 | (37%) | 7 | (35%) |
| HCV^{b} (%) | 0 | (0%) | 7 | (35%) |
| HBV^{c} (%) | 0 | (0%) | 2 | (10%) |
| Primary biliary | 2 | (11%) | 0 | (0%) |
| cholangitis (%) | | | | |
| Primary Sclerosing | 1 | (5%) | 0 | (0%) |
| Cholangitis (%) | | | | |
| Autoimmune hepatitis | 1 | (5) | 0 | (0%) |
| (%) | | | | |
| Other (%) | 0 | (0%) | 0 | (0%) |
| Child-Pugh Score | | | | |
| 5-6 | 8 | | 8 | |
| 7-9 | 10 | | 1 | |
| 10-15 | 1 | | 1 | |
| Diabetes Mellitus (%) | 9 | (47%) | 10 | (50%) |
| Hypertension (%) | 6 | (32%) | 16 | (80%) |
| Coronary Artery | 2 | (11%) | 7 | (35%) |
| Disease (%) | | | | |
| Hyperlipidemia (%) | 5 | (26%) | 14 | (70%) |
| Psychiatric Disorder | 3 | (16%) | 6 | (30%) |
| (%) | | | | |
| Other Cancer (%) | 1 | (5%) | 3 | (15%) |
| COPD^{d}/Asthma/OSA^{e} | 3 | (16%) | 6 | (30%) |
| Thyroid | 5 | (26%) | 0 | (0%) |
| Other PH^{f} | 0 | (0%) | 0 | (0%) |
| Ascites | 8 | (42%) | 1 | (5%) |
| Encephalopathy | 8 | (42%) | 0 | (0%) |
| Mean Hemoglobin | 10.68 | (0.7) | 12.80 | (0.5) |
| (std. err) | | | | |
| Mean Platelets | 116 | (16.2) | 210 | (19.3) |
| (std. err) | | | | |
| Mean ALP^{g} | 156.45 | (29.5) | 162.85 | (40.0) |
| (std. err) | | | | |
| Mean AST^{h} | 54.53 | (8.4) | 56.30 | (7.4) |
| (std. err) | | | | |
| Mean ALTⁱ | 34.79 | (6.8) | 52.20 | (7.0) |
| (std. err) | | | | |
| Mean Bilirubin | 1.99 | (0.4) | 0.91 | (0.2) |
| (std. err) | | | | |
| Mean Albumin | 3.42 | (0.1) | 3.80 | (0.1) |
| (std. err) | | | | |
| Mean INR^{j} | 1.26 | (0.06) | 1.14 | (0.05) |
| (std. err) | | | | |
| Mean Creatinine | 1.10 | (0.1) | 1.18 | (0.2) |
| (std. err) | | | | |

^{a}Alcoholic hepatitis.
^{b}Hepatitis C.
^{c}Hepatitis B.
^{d}Chronic Obstructive Pulmonary Disease.
^{e}Obstructive Sleep Apnea.
^{f}Pulmonary Hypertension
^{g}Alkaline Phosphatase.
^{h}Aspartate Aminotransferase.
ⁱAlanine Transamnase.
^{j}International Normalized Ratio.

### Small RNA-seq Extraction and Sequencing

Small RNA library preps were prepared using the QIAseq miRNA Library Kit (QIAGEN). Adapters are first ligated sequentially to the 3′ and 5′ end of the miRNAs followed by cDNA synthesis with UMI assignment, cDNA cleanup, library amplification and final library cleanup. All protocol steps were followed based on the use of the miRNeasy Serum/Plasma kit used upstream for purification of RNA, which has been shown to be effective for saliva samples (see e.g., Zahran F, et al. Oral Diseases. 21:739-747 (2015)). The starting amount of total RNA was 5 ul of the RNA eluate when 200 ul of sample has been processed using the miRNAeasy Serum/Plasma Kit. Adapter dilutions throughout the protocol followed the Serum/Plasma recommendations. Cycles of library amplification followed that of a 10 ng input sample with 19 total cycles. Two RNA control samples (Human XpressRef RNA, QIAGEN) with an input of 10 ng RNA were processed alongside the saliva samples. Final libraries were validated by Qubit Fluorometer (Invitrogen) and Fragment Analyzer (Agilent Technologies, Inc), and quantified via qPCR using NEBNext Library Quant Kit for Illumina (New England BioLabs, Inc). Pooled libraries were diluted, denatured and loaded onto the Illumina NextSeq 550 System, following the NextSeq User Guide. All 42 libraries were sequenced on one NextSeq High Output flow cell, single read 75 cycle run. FastQ files were developed for downstream analysis.

### Data Processing

FastQ files were first evaluated for read quality, using FastQC v0.11 (see e.g., Andrews S. FastQC: a quality control tool for high throughput sequence data. Babraham Bioinformatics, Babraham Institute, Cambridge, United Kingdom; 2010). Individual reads were then trimmed for phred quality scores (Q>20) and presence of adapters, using fastp v0.19 software (Chen S, et al. (2018) Bioinformatics 34:884-i890). Processed FastQ files were then aligned to the human genome (hg38) (Schneider V A, et al. (2017) Genome research. 27:849-864) and annotated using known miRNA sequences from miRBase v22 (Griffiths-Jones S. (2010) Current protocols in bioinformatics 29:12-9). Alignments and miRNA counts were performed using the Rsubread package v2.2 (Liao Y, et al. (2019) Nucleic Acids Research 47:e47-e47) following the authors' guidelines for miRNA.

### Differential Expression Analysis

Expression of miRNA was compared between HCC and cirrhosis samples using the R package DESeq2 v1.28 (Love M I, et al. (2014) Genome biology 15:550). First, raw read counts were normalized to account for sequencing depth and RNA composition (Anders S, et al. Nature Precedings. 2010;1-1). Wald's test was then performed on the normalized counts to identify any differentially expressed miRNA, using the cirrhosis samples as reference. In addition, patients were stratified to perform comparisons between those with chronic liver disease and the cirrhosis reference samples to distinguish miRNA associations specific to HCC versus those that may be due to impaired liver function. Log₂ fold changes (log FC) were estimated using empirical Bayes procedure described in Zhu et al. (2019) and implemented with the R package apeglm v1.10 (Zhu A, et al. (2019) Bioinformatics 35:2084-2092). All P values were false discovery rate (FDR) corrected using the Benjamini-Hochberg method (Benjamini Y, et al. Journal of the Royal Statistical Society. Series B (Methodological). 1995; 57:289-300). Any miRNA with an FDR P<0.05 was considered significantly differentially expressed. Principal components analysis (PCA) was used to identify any clustering between samples.

Comparison to Tissue-Based miRNA Profiles

Differentially abundant miRNAs were compared to microarray expression data from Martinez-Quetglas et al. (2016), GEO Accession Number: GSE74618 (Martinez-Quetglas I, et al. (2016) Gastroenterology 151:1192-1205). These data included miRNA expression data from 218 samples from human HCC tumors and 10 samples from cirrhotic non-tumoral tissue. Differential expression was conducted on the normalized expression values between HCC and cirrhosis samples, using the GEO2R tool available from the GEO database (Barrett T, Wilhite et al. (2012) Nucleic acids research 41:D991-D995). Overlapping miRNA detected in both GSE74618 and the salivary miRNA data were compared and were adjusted using a FDR approach, as described above.

### Predictive Modeling for Biomarker Development

Significant miRNA (FDR P<0.05) were assessed on their ability to differentiate between HCC and cirrhosis samples. Least absolute shrinkage and selection operator (LASSO; Tibshirani R. (1996) Journal of the Royal Statistical Society: Series B (Methodological) 58:267-288) with an optimized lambda of 0.14 was first used to select miRNA. Normalized expression values for each miRNA were then used to predict disease status, either HCC or cirrhosis, in logistic regression models. Ten-fold cross-validation was used when training each model to limit overfitting. Model accuracy was assessed based on the area under the receiver operating characteristic curve (AUC), sensitivity, specificity, positive predictive value (PPV), negative predictive value (NPV), and balanced accuracy. PPV is the ratio of true positives out of all identified positives, whereas NPV is the ratio of true negatives out of all identified negatives.

### Result

Differential Expression of miRNAs in the Saliva of HCC Patients

A total of 4,565 miRNAs were detected and 695 miRNAs were significantly differentially expressed (FDR P<0.05) between HCC and cirrhosis samples, demonstrating a broad shift in miRNA profiles.

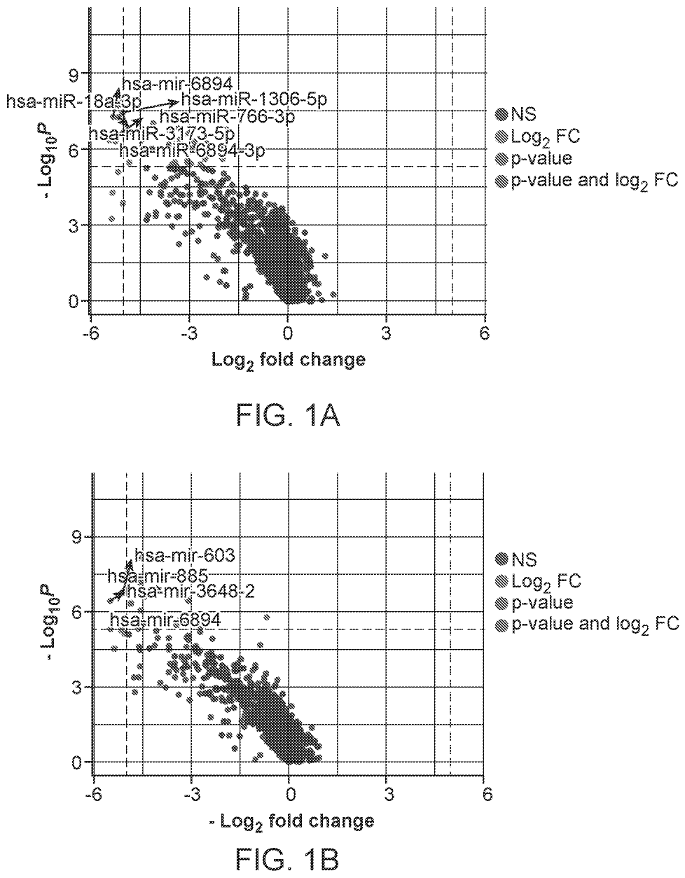

The majority of these miRNA, 621, were downregulated (log FC<0) in the HCC samples compared to cirrhosis samples, and 158 of those had a log FC<−2 (FIG. 1). The five most significantly differentially expressed miRNA included: mir-10401 (log FC=−8.78; FDR P=2.50×10⁻⁸), mir-3648-1 (log FC=−8.19; FDR P=6.14×10⁻¹¹), miR-10401-3p (log FC=−8.40; FDR P=2.66×10⁻⁷), mir-3648-2 (log FC=−7.16; FDR P=5.22×10⁻⁷), and miR-4492 (log FC=−8.24; FDR P=4.73×10⁻⁶). The 20 most significantly differentially expressed miRNA are shown in Table 2.

TABLE 2
Results for 20 most significantly differentially expressed miRNA.

| | | Log₂ | | FDR P |
|---|---|---|---|---|
| miRNA | Log₂ FC | FC SE | P Value | Value |
| hsa-mir-10401 | −8.78 | 1.30 | 1.51 × 10⁻¹¹ | 2.50 × 10⁻⁸ |
| hsa-mir-3648-1 | −8.20 | 1.25 | 6.14 × 10⁻¹¹ | 5.09 × 10⁻⁸ |
| hsa-miR-10401-3p | −8.40 | 1.35 | 4.82 × 10⁻¹⁰ | 2.66 × 10⁻⁷ |
| hsa-mir-3648-2 | −7.16 | 1.18 | 1.26 × 10⁻⁹ | 5.22 × 10⁻⁷ |
| hsa-miR-4492 | −8.24 | 1.45 | 1.43 × 10⁻⁸ | 4.73 × 10⁻⁶ |
| hsa-miR-18a-3p | −5.46 | 0.97 | 1.85 × 10⁻⁸ | 5.10 × 10⁻⁶ |
| hsa-miR-1306-5p | −5.56 | 1.01 | 3.74 × 10⁻⁸ | 8.85 × 10⁻⁶ |
| hsa-mir-6894 | −5.73 | 1.05 | 5.36 × 10⁻⁸ | 1.01 × 10⁻⁵ |
| hsa-miR-3173-5p | −5.48 | 1.01 | 5.49 × 10⁻⁸ | 1.01 × 10⁻⁵ |
| hsa-mir-1306 | −4.86 | 0.91 | 9.56 × 10⁻⁸ | 1.58 × 10⁻⁵ |
| hsa-mir-6806 | −6.10 | 1.16 | 1.51 × 10⁻⁷ | 1.91 × 10⁻⁵ |
| hsa-miR-93-3p | −3.79 | 0.72 | 1.53 × 10⁻⁷ | 1.91 × 10⁻⁵ |
| hsa-miR-3614-5p | −4.60 | 0.88 | 1.70 × 10⁻⁷ | 1.91 × 10⁻⁵ |
| hsa-mir-3615 | −3.98 | 0.76 | 1.73 × 10⁻⁷ | 1.91 × 10⁻⁵ |
| hsa-miR-3615 | −3.98 | 0.76 | 1.73 × 10⁻⁷ | 1.91 × 10⁻⁵ |
| hsa-mir-4449 | −5.70 | 1.10 | 2.06 × 10⁻⁷ | 2.13 × 10⁻⁵ |
| hsa-miR-942-5p | −3.49 | 0.68 | 2.48 × 10⁻⁷ | 2.42 × 10⁻⁵ |
| hsa-miR-25-5p | −4.95 | 0.96 | 2.78 × 10⁻⁷ | 2.55 × 10⁻⁵ |
| hsa-miR-12136 | −3.54 | 0.70 | 4.22 × 10⁻⁷ | 3.48 × 10⁻⁵ |
| hsa-miR-766-3p | −5.94 | 1.18 | 4.41 × 10⁻⁷ | 3.48 × 10⁻⁵ |

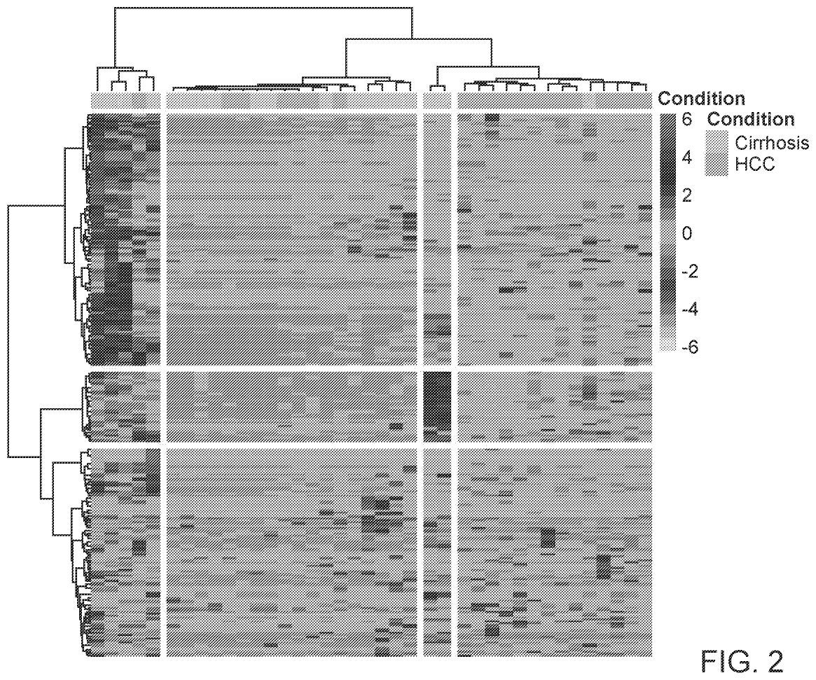

A heatmap showing the expression of the significant miRNA (FDR P<0.05 and absolute |log FC|>1) are shown in FIG. 2. Interestingly, a cluster of majority HCC patients was identified (n=13 out of 14). The other three clusters had cirrhosis patients in majority. The number of cirrhosis patients in the three clusters was 2 (100%), 4 (80%) and 12(66.7%), respectively. The cluster which consisted of 80% cirrhosis patients demonstrated a large relative increase across many miRNAs compared to those with HCC (FIG. 2).

Comparison Between Patients with and without Chronic Liver Disease

Eleven out of twenty patient samples with HCC also had chronic liver disease. These samples were also compared with cirrhosis samples but no HCC. This resulted in 468 differentially expressed miRNAs, of which 441 (94.23%) were also significant in the analysis between all HCC samples and those with cirrhosis only, suggesting that these differentially expressed miRNAs are likely to be due to the presence of HCC rather than the presence or lack of chronic liver disease. All 441 miRNA were downregulated (log FC<0) in the HCC samples with chronic liver disease compared to those only cirrhosis, and 400 of these had a log FC<−2 (FIG. 1B). The five most significantly differentially expressed miRNA included: miR-761 (log FC=−6.73; FDR P=8.54×10⁻⁸), mir-10401 (log FC=−9.35; FDR P=5.72×10⁻⁷), mir-3648-1 (log FC=−8.74; FDR P=2.39×10⁻⁶), miR-12136 (log FC=4.79; FDR P=2.39×10⁻⁶), and miR-10401-3p-(log FC=−8.98; FDR P=4.76×10⁻⁶).

Comparison to Tissue-Based miRNA

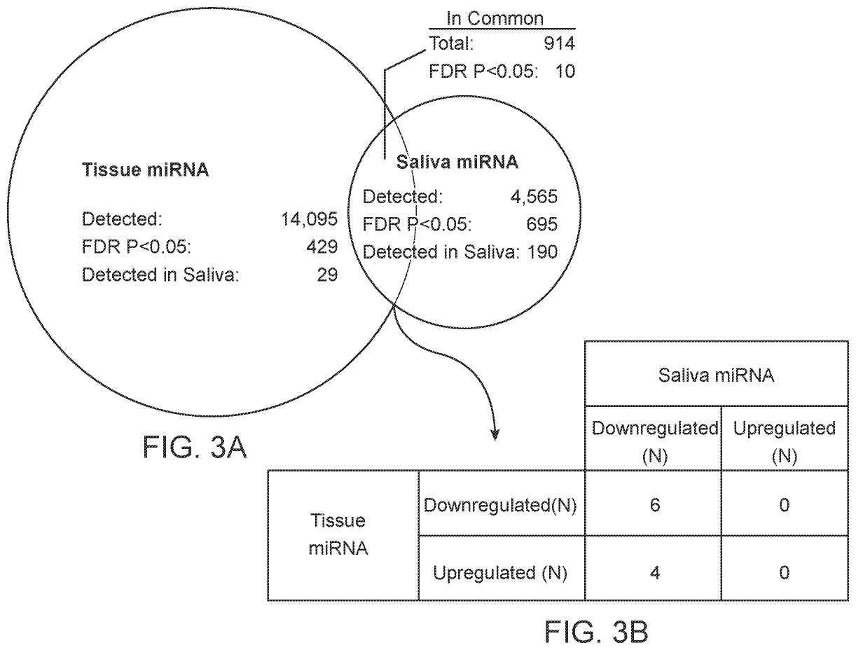

Ten significant miRNAs detected in saliva were also found to be differentially expressed in HCC tissue samples compared to cirrhotic liver tissue samples (FDR P<0.05) (NIH Gene Expression Omnibus (GSE74618), Martinez-Quetglas et al. (2016) Gastroenterology 151:1192-1205, FIG. 3 and Table 4). 429 miRNA were significantly different between HCC and cirrhosis tissue samples (FDR P<0.05), 29 of which were detected in the saliva; whereas out of the 695 significant salivary miRNA, 190 were also detected in tissue. However, only 10 miRNAs were significantly different in both cohorts (FDR P<0.05) (FIG. 3). Out of the 10 significant miRNAs found in each dataset, all were downregulated in saliva, but 6 were downregulated and 4 were upregulated in tissue (FIG. 3 and Table 3).

TABLE 3
Results for 20 most significant common miRNAs in saliva compared
to tissue samples from Martinez-Quetglas et al. (2016).

| | HCC vs Cirrhosis Liver Tissue^{a} | | | HCC vs Cirrhosis Saliva | |
|---|---|---|---|---|---|
| Core miRNA | miRNA^{b} | logFC | FDR P Value | logFC | FDR P Value |
| hsa-mir-1306 | hsa-miR-1306_st | −0.045 | 7.77E−01 | −4.09 | 1.58E−05 |
| hsa-mir-1306 | hp_hsa-mir-1306_st | −0.0179 | 9.25E−01 | −4.09 | 1.58E−05 |
| hsa-mir-125b-1 | hp_hsa-mir-125b-1_x_st | −0.286 | 2.05E−03 | −2.47 | 3.66E−05 |
| hsa-mir-125b-2 | hp_hsa-mir-125b-2_st | −0.212 | 9.77E−03 | −2.47 | 3.66E−05 |
| hsa-mir-125b-2 | hp_hsa-mir-125b-2_x_st | −0.187 | 1.26E−01 | −2.47 | 3.66E−05 |
| hsa-mir-2110 | hsa-miR-2110_st | −0.296 | 3.02E−01 | −3.60 | 3.66E−05 |
| hsa-mir-2110 | hp_hsa-mir-2110_st | −0.0296 | 8.92E−01 | −3.60 | 3.66E−05 |
| hsa-mir-125b-1 | hp_hsa-mir-125b-1_st | −0.0132 | 9.51E−01 | −2.47 | 3.66E−05 |
| hsa-mir-320d-2 | hp_hsa-mir-320d-2_x_st | −0.133 | 3.15E−01 | −3.10 | 4.02E−05 |
| hsa-mir-92a-1 | hp_hsa-mir-92a-1_x_st | 0.0615 | 7.30E−01 | −2.06 | 4.13E−05 |
| hsa-mir-92a-1 | hp_hsa-mir-92a-1_st | −0.00649 | 9.80E−01 | −2.06 | 4.13E−05 |
| hsa-mir-92a-2 | hp_hsa-mir-92a-2_x_st | −0.117 | 4.25E−01 | −2.02 | 4.35E−05 |
| hsa-mir-92a-2 | hp_hsa-mir-92a-2_st | −0.0468 | 8.03E−01 | −2.02 | 4.35E−05 |
| hsa-mir-942 | hp_hsa-mir-942_st | −0.113 | 2.33E−01 | −2.89 | 4.46E−05 |
| hsa-mir-942 | hsa-miR-942_st | 0.0492 | 6.58E−01 | −2.89 | 4.46E−05 |
| hsa-mir-766 | hp_hsa-mir-766_st | −0.0191 | 9.12E−01 | −4.16 | 4.81E−05 |
| hsa-mir-766 | hsa-miR-766_st | 0.026 | 9.53E−01 | −4.16 | 4.81E−05 |
| hsa-mir-3173 | hsa-miR-3173_st | 0.0865 | 5.66E−01 | −4.37 | 6.22E−05 |
| hsa-mir-3173 | hp_hsa-mir-3173_st | −0.00754 | 9.78E−01 | −4.37 | 6.22E−05 |
| hsa-mir-10b | hsa-miR-10b_st | 1.21 | 1.27E−01 | −3.03 | 0.00012291 |
| hsa-mir-10b | hp_hsa-mir-10b_st | 0.0476 | 7.52E−01 | −3.03 | 0.00012291 |
| hsa-mir-10b | hsa-miR-10b-star_st | 0.0532 | 8.67E−01 | −3.03 | 0.00012291 |
| hsa-mir-885 | hsa-miR-885-3p_st | 1.04 | 1.48E−01 | −3.39 | 0.00014375 |
| hsa-mir-885 | hsa-miR-885-5p_st | −0.892 | 4.30E−01 | −3.39 | 0.00014375 |
| hsa-mir-885 | hp_hsa-mir-885_st | 0.0999 | 7.75E−01 | −3.39 | 0.00014375 |
| hsa-mir-671 | hsa-miR-671-3p_st | 0.153 | 2.15E−01 | −2.42 | 0.0001755 |
| hsa-mir-671 | hsa-miR-671-5p_st | 0.405 | 6.12E−01 | −2.42 | 0.0001755 |
| hsa-mir-122 | hsa-miR-122-star_st | −0.351 | 6.22E−01 | −3.45 | 0.0001755 |
| hsa-mir-671 | hp_hsa-mir-671_st | 0.0591 | 6.85E−01 | −2.42 | 0.0001755 |
| hsa-mir-122 | hp_hsa-mir-122_st | −0.0381 | 7.79E−01 | −3.45 | 0.0001755 |
| hsa-mir-122 | hsa-miR-122_st | −0.149 | 7.94E−01 | −3.45 | 0.0001755 |

^{a}Data obtained from NIH Gene Expression Omnibus (GSE74618), previously published by Martinez-Quetglas et al. (2016).
^{b}miRNA identifier reported in Martinez-Quetglas et al. (2016).

The miRNA comparisons are provided in Table 4. The most significant miRNA common to both datasets were hsa-mir-125b-2 (FDR P=3.66×10⁻⁵), hsa-mir-125b-1 (FDR P=3.66×10−5) and hsa-mir-106b (FDR P=8.11×10⁻⁴).

TABLE 4
Comparisons between saliva and tissue miRNA significantly
altered in HCC versus cirrhosis.

| | miRNA significant downregulated in |
|---|---|
| miRNA significant downregulated | HCC saliva and upregulated in HCC |
| in HCC saliva and tissue^{a} | tissue^{a} (n = 18) |
| hsa-mir-125b-1 | hsa-mir-106b |
| hsa-mir-125b-2 | hsa-mir-2277 |
| hsa-mir-139 | hsa-mir-3180-1 |
| hsa-mir-375 | hsa-mir-93 |
| hsa-mir-548i-2 | |
| hsa-mir-548l | |

^{a}Data obtained from NIH Gene Expression Omnibus (GSE74618), previously published by Martinez-Quetglas et al. (2016) Gastroenterology 151: 1192-1205.

Saliva miRNA Predictive Modeling

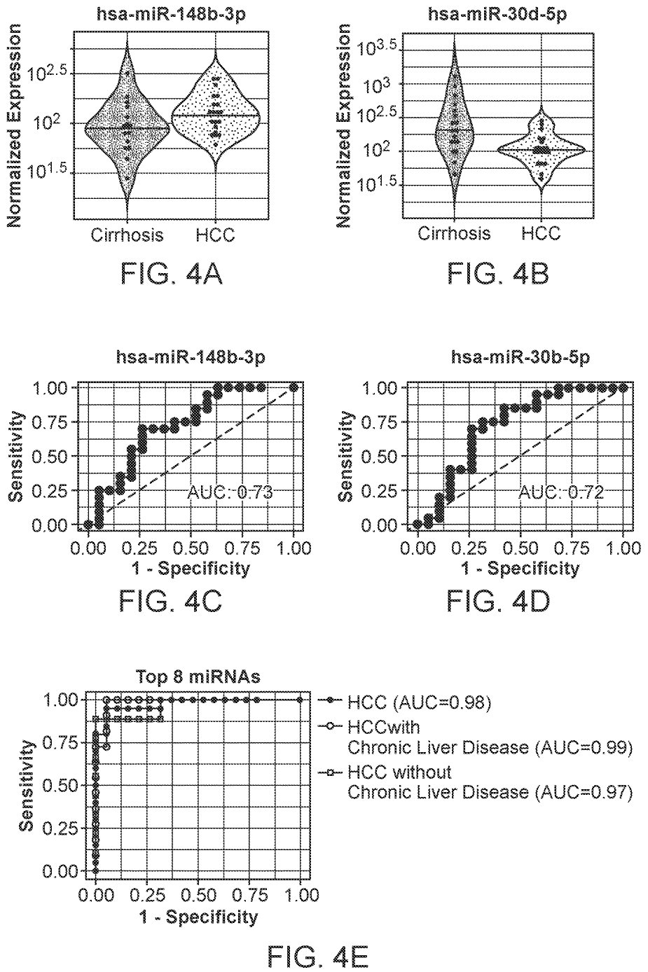

Eight miRNAs were selected using LASSO: hsa-miR-30d-5p, hsa-miR-148b-3p, hsa-mir-6806, hsa-miR-126-3p, hsa-miR-193a-3p, hsa-miR-8059, hsa-miR-6512-5p and hsa-mir-505. Table 5 shows the AUC, sensitivity, specificity, and balanced accuracy for each model, as well as a combined model, utilizing all 8 miRNAs. The three miRNAs with the highest accuracy included hsa-miR-148-3p (AUC=0.73), hsa-miR-30d-5p (AUC=0.72), and mir-6806 (AUC=0.71). Each displayed equal specificity, or true negative rate of 0.74. The sensitivities were 0.65, 0.70, and 0.70 for hsa-mir-6806, hsa-miR-148b-3p, and hsa-miR30d-5p, respectively. The combined model (AUC=0.98) displayed the highest balanced accuracy, 0.95. The corresponding sensitivity and specificity were 0.95 and 0.95, respectively. Comparing only samples with chronic liver disease provided no evidence of the model being biased toward HCC samples without chronic liver disease, and the ROC curves overlapped substantially (FIG. 4) and the cross-validated AUC values for subset of HCC with chronic liver disease and HCC without chronic liver disease were 0.99 and 097, respectively.

TABLE 5
Accuracy metrics for predicting HCC from cirrhosis.

| | | | | | Balanced | |
|---|---|---|---|---|---|---|
| miRNA | Sensitivity | Specificity | PPV | NPV | Accuracy | AUC |
| All 8 Selected miRNA | 0.95 | 0.95 | 0.95 | 0.95 | 0.95 | 0.98 |
| hsa-mir-148b-3p | 0.70 | 0.74 | 0.74 | 0.70 | 0.72 | 0.73 |
| hsa-mir-30d-5p | 0.70 | 0.74 | 0.70 | 0.74 | 0.72 | 0.72 |
| hsa-mir-6806 | 0.65 | 0.74 | 0.72 | 0.67 | 0.69 | 0.71 |
| hsa-mir-6512-5p | 0.50 | 0.89 | 0.89 | 0.60 | 0.70 | 0.69 |
| hsa-mir-126-3p | 0.95 | 0.42 | 0.63 | 0.89 | 0.69 | 0.63 |
| hsa-mir-505 | 0.95 | 0.37 | 0.61 | 0.88 | 0.66 | 0.57 |
| hsa-mir-8059 | 1.00 | 0.26 | 0.59 | 1.00 | 0.63 | 0.48 |
| hsa-mir-193a-3p | 0.40 | 0.95 | 0.89 | 0.60 | 0.67 | 0.66 |

Table 6 (below) shows mean counts of miRNA reads from the next generation sequencing platform for each disease group.

TABLE 6
miRNA Mean and Standard Deviation by Group

| | | | HCC without | | | | | |
|---|---|---|---|---|---|---|---|---|
| | | | chronic liver | | HCC with chronic | | | |
| | Cirrhosis | | disease | | liver disease | | All HCC | |
| miRNA | Mean (Std. Dev.) | | Mean (Std. Dev.) | | Mean (Std. Dev.) | | Mean (Std. Dev.) | |
| hsa-miR-148b-3p | 508.21 | (708.58) | 442.78 | (404.58) | 809.36 | (622.02) | 644.4 | (554.58) |
| hsa-miR-193a-3p | 0.68 | (1.49) | 0.78 | (1.09) | 1.27 | (1.95) | 1.05 | (1.61) |
| hsa-mir-8059 | 2.84 | (4.26) | 1.22 | (1.09) | 1.36 | (1.63) | 1.3 | (1.38) |
| hsa-mir-6806 | 1.16 | (1.01) | 0.67 | (0.87) | 0.36 | (0.67) | 0.5 | (0.76) |
| hsa-miR-6512-5p | 2.11 | (4.52) | 1.78 | (1.86) | 2.09 | (2.02) | 1.95 | (1.9) |
| hsa-miR-30d-5p | 1394.74 | (2168.7) | 559.22 | (561.27) | 860.73 | (781.88) | 725.05 | (691.44) |
| hsa-miR-126-3p | 389.68 | (1382.96) | 43.22 | (43) | 110.82 | (100.45) | 80.4 | (85.32) |
| hsa-mir-505 | 23.84 | (31.13) | 16.33 | (15.76) | 25.91 | (23.74) | 21.6 | (20.62) |

PPV: positive predictive value; NPV: negative predictive value; AUC: Area under the receiver operating characteristic curve.

Algorithm 1 (below) may be used to evaluate the risk of HCC in an individual, based on differential expression of the eight selected miRNAs.

Ŷ˜−5.371+0.110(hsa-miR-148b-3p)+5.550(hsa-miR-193a-3p)+−0.441(hsa-mir-8059)+−1.281(hsa-mir-6806)+1.700(hsa-miR-6512-5p)+−0.018(hsa-miR-30d-5p)+−0.037(hsa-miR-126-3p)+0.051(hsa-mir-505)
Ŷ=0.44 Indicates elevated risk of HCC with performance as indicated in Table 7.

Users may select a threshold in order to tailor the performance to their specific tolerance of allowing false positive or false negative predictions of HCC. A higher threshold could be selected to increase the confidence that detected positives truly have HCC, but at the risk of increasing false negative predictions. Conversely, one could select a lower threshold in order to minimize the number of patients missed that have truly HCC (true positives), but will result in additional false positive predictions. Here, we identified an optimal threshold of 0.43 in order to maximize both sensitivity and specificity.

TABLE 7
Model performance of Algorithm 1 across various Ŷ thresholds.

| | | | | | | | Model |
|---|---|---|---|---|---|---|---|
| | | | | | | | Area |
| | | | | | | Balanced | Under the |
| Ŷ Threshold | Sensitivity | Specificity | PPV^{a} | NPV^{b} | Precision | Accuracy | Curve |
| 0.126 | 0.95 | 0.68 | 0.76 | 0.93 | 0.76 | 0.82 | 0.98 |
| 0.157 | 0.95 | 0.74 | 0.79 | 0.93 | 0.79 | 0.84 | |
| 0.212 | 0.95 | 0.79 | 0.83 | 0.94 | 0.83 | 0.87 | |
| 0.248 | 0.95 | 0.84 | 0.86 | 0.94 | 0.86 | 0.90 | |
| 0.272 | 0.95 | 0.89 | 0.90 | 0.94 | 0.90 | 0.92 | |
| 0.438^{c} | 0.95 | 0.95 | 0.95 | 0.95 | 0.95 | 0.95 | |
| 0.670 | 0.90 | 0.95 | 0.95 | 0.90 | 0.95 | 0.92 | |
| 0.789 | 0.85 | 0.95 | 0.94 | 0.86 | 0.94 | 0.90 | |
| 0.824 | 0.80 | 0.95 | 0.94 | 0.82 | 0.94 | 0.87 | |
| 0.859 | 0.80 | 1.00 | 1.00 | 0.83 | 1.00 | 0.90 | |

^{a}positive predictive value;
^{b}negative predictive value;
^{c}optimal threshold based on balanced accuracy

TABLE 8
Ranges for model coefficients (β).

| | Coefficients (β) | Standard Error | Coefficient Range |
|---|---|---|---|
| (Intercept) | −5.371 | 4.006 | −13.383-2.64 |
| hsa-miR-148b-3p | 0.111 | 0.056 | −0.001-0.222 |
| hsa-miR-193a-3p | 5.550 | 8.533 | −11.516-22.615 |
| hsa-mir-8059 | −0.441 | 0.910 | −2.261-1.379 |
| hsa-mir-6806 | −1.281 | 0.919 | −3.12-0.557 |
| hsa-miR-6512-5p | 1.700 | 2.914 | −4.128-7.528 |
| hsa-miR-30d-5p | −0.018 | 0.008 | −0.035-−0.001 |
| hsa-miR-126-3p | −0.037 | 0.036 | −0.11-0.036 |
| hsa-mir-505 | 0.051 | 0.090 | −0.128-0.23 |

Certain modifications and improvements will occur to those skilled in the art upon a reading of the foregoing description. It should be understood that all such modifications and improvements have been deleted herein for the sake of conciseness and readability but are properly within the scope of the following claims.

What is claimed is:

1. A method for diagnosing or prognosticating hepatocellular carcinoma (HCC) in a subject, or for assessing the risk of developing hepatocellular carcinoma, or for monitoring the effectiveness of an anti-tumor therapy against hepatocellular carcinoma, comprising determining, in an isolated sample of saliva, the expression level of at least one microRNA (miRNA) having at least 90% sequence identity with an miRNA selected from the group comprising or consisting of hsa-mir-148b-3p, hsa-mir-148b-5p, hsa-mir-30d-3p, hsa-mir-30d-5p, hsa-mir-6806-3p, hsa-mir-6806-5p, hsa-mir-6512-3p, hsa-mir-6512-5p, hsa-mir-126-3p, hsa-mir-126-5p, hsa-mir-505-3p, hsa-mir-505-5p, hsa-mir-8059, hsa-mir-193a-3p, and hsa-mir-193a-5p and determining whether the miRNA in the saliva sample is differentially expressed as compared to as compared to a reference saliva sample, wherein the differential expression of miRNA is an upregulation or a downregulation of miRNA expression, in order to determine whether the subject has HCC, is at elevated risk of having HCC, or is receiving effective treatment for HCC, and treating the subject diagnosed with HCC or having elevated risk of having HCC, or as needing further effective treatment for HCC with a compound or other therapy to improve the HCC.

2. The method of claim 1, wherein the reference sample is a sample from a subject having cirrhosis.

3. The method of claim 1, wherein differential expression is detected for at least one miRNA selected from the group comprising or consisting of hsa-miR-30d-5p, hsa-miR-148b-3p, hsa-mir-6806, and hsa-miR-126-3p as compared to the reference sample.

4. The method of claim 3, wherein the at least one miRNA is selected from the group comprising hsa-miR-30d-5p, hsa-miR-148b-3p, hsa-mir-6806, and hsa-miR-126-3p, and optionally further comprising one or more of hsa-mir-6512-5p, hsa-mir-505, hsa-mir-8059, and/or hsa-mir-193a-3p.

5. The method of claim 4, wherein the at least one miRNA is selected from the group consisting of hsa-mir-148b-3p, hsa-mir-30d-5p, hsa-mir-6806, hsa-mir-6512-5p, hsa-mir-126-3p, hsa-mir-505, hsa-mir-8059, and hsa-mir-193a-3p as compared to a reference expression sample.

6. The method of claim 5, wherein normalized read counts for each miRNA from a small RNA-seq assay are analyzed according to the formula:
Ŷ˜−5.371+0.110(hsa-miR-148b-3p)+5.550(hsa-miR-193a-3p)+−0.441(hsa-mir-8059)+−1.281(hsa-mir-6806)+1.700(hsa-miR-6512-5p)+−0.018(hsa-miR-30d-5p)+−0.037(hsa-miR-126-3p)+0.051(hsa-mir-505)
wherein Ŷ=0.44 indicates the subject is at elevated risk of HCC.

7. The method of claim 6, wherein the method has sensitivity of at least 95% and specificity of at least 95%.

8. The method of claim 6, wherein the method has an accuracy of at least 95%

9. The method of any one of claims 1 to 8, wherein the reference sample is from a subject who does not have HCC.

10. The method of claim 9, wherein the reference sample is from a subject who has cirrhosis.

11. A method for differentiating a subject having hepatocellular carcinoma (HCC) from a subject having liver cirrhosis, the method comprising the step of determining, in an isolated sample of saliva, the expression level of at least one microRNA (miRNA) having at least 90% sequence identity with an miRNA selected from the group comprising or consisting of hsa-mir-148b-3p, hsa-mir-148b-5p, hsa-mir-30d-3p, hsa-mir-30d-5p, hsa-mir-6806-3p, hsa-mir-6806-5p, hsa-mir-6512-3p, hsa-mir-6512-5p, hsa-mir-126-3p, hsa-mir-126-5p, hsa-mir-505-3p, hsa-mir-505-5p, hsa-mir-8059, hsa-mir-193a-3p, and hsa-mir-193a-5p and determining whether the miRNA in the saliva sample is differentially expressed as compared to a reference saliva sample, wherein differential expression of the miRNA indicates the subject has HCC.

12. The method of claim 11, wherein the at least one miRNA is selected from the group comprising or consisting of hsa-miR-30d-5p, hsa-miR-148b-3p, hsa-mir-6806, and hsa-miR-126-3p.

13. The method of claim 12, wherein the at least one miRNA is selected from the group comprising hsa-miR-30d-5p, hsa-miR-148b-3p, hsa-mir-6806, and hsa-miR-126-3p, and optionally further comprising one or more of hsa-mir-6512-5p, hsa-mir-505, hsa-mir-8059, and/or hsa-mir-193a-3p.

14. The method of claim 13, wherein the at least one miRNA is selected from the group consisting of: hsa-mir-148b-3p, hsa-mir-30d-5p, hsa-mir-6806, hsa-mir-6512-5p, hsa-mir-126-3p, hsa-mir-505, hsa-mir-8059, and hsa-mir-193a-3p.

15. The method of claim 14, wherein normalized read counts for each miRNA from a small RNA-seq assay are analyzed according to the formula:
Ŷ˜−5.371+0.110(hsa-miR-148b-3p)+5.550(hsa-miR-193a-3p)+−0.441(hsa-mir-8059)+−1.281(hsa-mir-6806)+1.700(hsa-miR-6512-5p)+−0.018(hsa-miR-30d-5p)+−0.037(hsa-miR-126-3p)+0.051(hsa-mir-505)
wherein Ŷ=0.44 indicates the subject has HCC.

16. The method of claim 14, wherein the method has sensitivity of at least 95% and specificity of at least 95%.

17. The method of claim 14, wherein the method has an accuracy of at least 95%.

18. A method for discovering miRNA biomarkers of hepatocellular carcinoma (HCC) in saliva from a subject having HCC, the method comprising:
(a) obtaining a saliva sample from the subject, and a saliva sample from a subject not having HCC, and
(b) detecting miRNAs that are differentially expressed in the subject having HCC compared to the subject not having HCC, wherein the differentially expressed miRNAs have a high predictive value for detection of HCC, and wherein the detection step comprises machine learning that utilizes least absolute shrinkage and selection operator (LASSO) and cross-validation, wherein differentially expressed miRNAs are miRNA biomarkers of HCC.

19. The method of claim 18, wherein the differentially expressed miRNA biomarkers of HCC are selected from the group comprising or consisting of hsa-mir-148b-3p, hsa-mir-30d-5p, hsa-mir-6806, hsa-mir-6512-5p, hsa-mir-126-3p, hsa-mir-505, hsa-mir-8059, and hsa-mir-193a-3p.

20. The method of claim 19, wherein the group of differentially expressed miRNA biomarkers comprises hsa-miR-30d-5p, hsa-miR-148b-3p, hsa-mir-6806, and hsa-miR-126-3p, and optionally further comprises one or more of hsa-mir-6512-5p, hsa-mir-505, hsa-mir-8059, and/or hsa-mir-193a-3p.

21. The method of claim 20, wherein the group of differentially expressed miRNA biomarkers consists of hsa-mir-148b-3p, hsa-mir-30d-5p, hsa-mir-6806, hsa-mir-6512-5p, hsa-mir-126-3p, hsa-mir-505, hsa-mir-8059, and hsa-mir-193a-3p.

22. The method of claim 21, wherein normalized read counts for each miRNA from a small RNA-seq assay are analyzed according to the formula:
Ŷ˜−5.371+0.110(hsa-miR-148b-3p)+5.550(hsa-miR-193a-3p)+−0.441(hsa-mir-8059)+−1.281(hsa-mir-6806)+1.700(hsa-miR-6512-5p)+−0.018(hsa-miR-30d-5p)+−0.037(hsa-miR-126-3p)+0.051(hsa-mir-505)
wherein Ŷ=0.44 indicates the subject has HCC.

23. The method of any of claims 18 to 21, wherein the subject not having HCC has liver cirrhosis.


SEQUENCE LISTING (16 sequences)

SEQ ID NO: 1 - RNA, 22 nt, Homo sapiens
ucagugcaucacagaacuuugu

SEQ ID NO: 2 - RNA, 22 nt, Homo sapiens
aaguucuguuauacacucaggc

SEQ ID NO: 3 - RNA, 22 nt, Homo sapiens
cuuucagucagauguuugcugc

SEQ ID NO: 4 - RNA, 22 nt, Homo sapiens
uguaaacauccccgacuggaag

SEQ ID NO: 5 - RNA, 22 nt, Homo sapiens
ugaagcucugacauuccugcag

SEQ ID NO: 6 - RNA, 24 nt, Homo sapiens
uguaggcaugaggcagggcccagg

SEQ ID NO: 7 - RNA, 64 nt, Homo sapiens
ugcucuguaggcaugaggcagggcccagguuccaugugaugcugaagcucugacauuccugcag

SEQ ID NO: 8 - RNA, 22 nt, Homo sapiens
uuccagcccuucuaaugguagg

SEQ ID NO: 9 - RNA, 22 nt, Homo sapiens
uaccauuagaagagcuggaaga

SEQ ID NO: 10 - RNA, 22 nt, Homo sapiens
ucguaccgugaguaauaaugcg

SEQ ID NO: 11 - RNA, 21 nt, Homo sapiens
cauuauuacuuuugguacgcg

SEQ ID NO: 12 - RNA, 22 nt, Homo sapiens
cgucaacacuugcugguuuccu

SEQ ID NO: 13 - RNA, 22 nt, Homo sapiens
gggagccaggaaguauugaugu

SEQ ID NO: 14 - RNA, 81 nt, Homo sapiens
uacaggugcaggggaacuguagaugaaaaggcuuggcacuugagggaaagccucaguucauucucauuuugcucaccuguu

SEQ ID NO: 15 - RNA, 22 nt, Homo sapiens
aacuggccuacaaagucccagu

SEQ ID NO: 16 - RNA, 22 nt, Homo sapiens
ugggucuuugcgggcgagauga